(19) United States
(12) Patent Application Publication
(10) Pub. No.: US 2003/0139646 A1
(43) Pub. Date: Jul. 24, 2003

(54) Devices and methods for manipulation of organ tissue

(76) Inventors: James S. Sharrow, Bloomington, MN (US); Frederick J. Foley, Bedford, NH (US); Thomas G. Adelman, West Baldwin, ME (US)

Correspondence Address: SHUMAKER & SIEFFERT, P. A., 8425 SEASONS PARKWAY, SUITE 105, ST. PAUL, MN, 55125, US

(21) Appl. No.: 10/161,267

(22) Filed: May 31, 2002

Related U.S. Application Data
(60) Provisional application No. 60/351,621, filed on Jan. 23, 2002.

(51) Int. Cl. A61F 2/00

(52) U.S. Cl. 600/37

(57) ABSTRACT

The invention provides techniques for holding a moving organ, such as a beating heart. A manipulation device that holds the organ includes an outer shell and an inner shell. Vacuum pressure applied to the outer shell draws the organ into the inner shell. The vacuum pressure is communicated to the inner shell chamber via one or more apertures in the inner shell. The inner shell may have a structure and a texture that enhances the hold on the organ, and the manipulation device may also include a skirt-like member to improve the seal between the manipulation device and the organ.

This application claims priority from U.S. Provisional Application Serial No. 60/351,621, filed Jan. 23, 2002, the entire content of which is incorporated herein by reference.

### TECHNICAL FIELD

The invention relates to devices capable of providing adherence to organs of the body for purposes of medical diagnosis and treatment. More particularly, the invention relates to devices capable of adhering to, holding, moving, stabilizing or immobilizing an organ.

### BACKGROUND

In many areas of surgical practice, it may be desirable to manipulate an internal organ without causing damage to the organ. In some circumstances, the surgeon may wish to turn, lift or otherwise reorient the organ so that surgery may be performed upon it. In other circumstances, the surgeon may simply want to move the organ out of the way. In still other cases, the surgeon may wish to hold the organ, or a portion of it, immobile so that it will not move during the surgical procedure.

Unfortunately, many organs are slippery and are difficult to manipulate. Holding an organ with the hands may be undesirable because of the slipperiness of the organ. Moreover, the surgeon's hands ordinarily cannot hold the organ and perform the procedure at the same time. The hands of an assistant may be bulky, becoming an obstacle to the surgeon. Also, manual support of an organ over an extended period of time can be difficult due to fatigue. Holding an organ with an instrument may damage the organ, especially if the organ is unduly squeezed, pinched or stretched. Holding an organ improperly may also adversely affect the functioning of the organ.

The heart is an organ that may be more effectively treated if it can be manipulated. Many forms of heart manipulation may be useful, including moving the heart within the chest and holding it in place. Some forms of heart disease, such as blockages of coronary vessels, may best be treated through procedures performed during open-heart surgery. During open-heart surgery, the patient is typically placed in the supine position. The surgeon performs a median sternotomy, incising and opening the patient's chest. Thereafter, the surgeon may employ a rib-spreader to spread the rib cage apart, and incise the pericardial sac to obtain access to the heart. For some forms of open-heart surgery, the patient is placed on cardiopulmonary bypass (CPB) and the patient's heart is arrested. Stopping the patient's heart is a frequently chosen procedure, as many coronary procedures are difficult to perform if the heart continues to beat. CPB entails trauma to the patient, with attendant side effects and risks. An alternative to CPB involves operating on the heart while the heart continues to beat.

Once the surgeon has access to the heart, it may be necessary to lift the heart from the chest or turn it to obtain access to a particular region of interest. Such manipulations are often difficult tasks. The heart is a slippery organ, and it is a challenging task to grip it with a gloved hand or an instrument without causing damage to the heart. Held improperly, the heart may suffer ischemia, hematoma or other trauma. The heart may also suffer a loss of hemodynamic function, and as a result may not pump blood properly or efficiently. Held insecurely, the heart may drop back into the chest, which may cause trauma to the heart and may interfere with the progress of the operation.

The problems associated with heart manipulation are greatly multiplied when the heart is beating. Beating causes translational motion of the heart in three dimensions. In addition, the ventricular contractions cause the heart to twist when beating. These motions of the heart make it difficult to lift the heart, move it and hold it in place. Moreover, the natural motions of the heart may cause the heart to disengage from a device designed to hold it.

### SUMMARY

In general, the invention provides techniques for holding a moving organ, such as a beating heart. A manipulation device that holds the organ includes an outer shell and an inner shell. The manipulation device holds the organ with vacuum pressure that draws the organ into the inner shell. The inner shell may have a structure and a texture that enhances the hold on the organ.

When a portion of the organ is placed within the chamber defined by the inner shell and vacuum pressure is applied to the outer shell, the vacuum pressure is communicated to the inner shell chamber via one or more apertures in the inner shell. In a typical embodiment, the inner shell may include a plurality of apertures. The apertures may be any shape, such as rectangular or circular. Each aperture may behave as if it were a source of suction, causing a significant fraction of the surface area of the organ to be brought in contact with the inner surface of the inner shell. Increased area of contact between the organ and the inner surface of the inner shell increases the frictional forces between the organ and the inner surface of the inner shell, thereby increasing adherence between the organ and the inner surface of the inner shell. In this way, adherence between the organ tissue and the inner shell is enhanced, the risk of slippage is reduced and the organ is held more securely.

In a representative application, the invention is directed to techniques for holding the apex of a beating heart. As the heart beats, the heart bobs and twists. The twisting is problematic for at least two reasons. First, the twisting is important for the proper hemodynamic functioning of the heart, and therefore simply restraining the heart from all rotational motion has undesirable consequences upon hemodynamic functions. Second, the twisting compounds the difficulty of holding the heart with the manipulation device. In addition, the weight and tension of the heart tends to pull the heart away from the manipulation device.

The invention is directed to techniques that reduce the chances that the heart tissue may twist or pull away from the manipulation device and may drop back into the chest or chafe against the manipulation device. In particular, the invention is directed to forming a gripping surface that contacts the heart and promotes a secure engagement between the heart and the inner shell. The engagement between the heart and the inner shell is enhanced by a combination of vacuum pressure and the texture of the inner surface of the inner shell. In this way, the heart is held without causing trauma or impairing hemodynamic functions.

In one embodiment, the invention is directed to an organ manipulation device. The device comprises an outer shell and an inner shell coupled to the outer shell. The inner and outer shells define a space and the inner shell defines a chamber. The inner shell includes at least one aperture in fluid communication with the space and the chamber. The device may further include a skirt-like member. The skirt-like member, which may improve the seal between the device and the organ, may be, for example, coupled to the outer shell, coupled to the inner shell, or formed integrally with the inner shell.

In another embodiment, the invention is directed to a method of making the organ manipulation device. The method comprises constructing an inner shell that defines a chamber, with the inner shell sized and shaped to fit inside an outer shell. The method also includes providing a aperture in the inner shell and coupling the inner shell to the outer shell to define a space. The aperture is in fluid communication with the space and the chamber.

In a further embodiment, the invention is directed to a method of using the organ manipulation device. The method comprises receiving an organ in a chamber defined by an inner shell, applying vacuum pressure to an outer shell coupled to the inner shell, the outer shell and the inner shell defining a space and applying vacuum pressure to the chamber via an aperture in fluid communication with the space and the chamber. An exemplary application of this method involves engaging an apex of a heart with the inner shell.

The invention can provide one or more advantages. For example, the inner shell helps to hold the organ more securely. The inner shell provides a large inner surface that contacts and grips the organ. The inner surface may be textured to improve the grip, and the apertures act like a plurality of suction sources that draw the tissue into and against the inner shell. The large contact area reduces the risk that the organ will accidentally slip out of the manipulation device. In the context of heart surgery, the invention helps the surgeon manipulate the heart and securely hold the heart in place. In addition, the invention may accommodate some translational and rotational motion of the heart, so that the hemodynamic functions of the heart are maintained. When vacuum pressure is discontinued, the organ may be disengaged from the manipulation device.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a perspective view of a manipulation device in conjunction with a beating heart.

FIG. 2 is a cross-sectional side view of an exemplary manipulation device.

FIG. 3 is a cross-sectional side view of an alternate exemplary embodiment of a manipulation device.

FIG. 4 is a cross-sectional side view of another exemplary embodiment of a manipulation device.

FIG. 5 is a cross-sectional side view of a further exemplary embodiment of a manipulation device.

FIG. 6 is a cross-sectional side view of an exemplary manipulation device similar to the device shown in FIG. 2.

FIG. 7 is a cross-sectional side view of an exemplary manipulation device similar to the device shown in FIG. 4.

### DETAILED DESCRIPTION

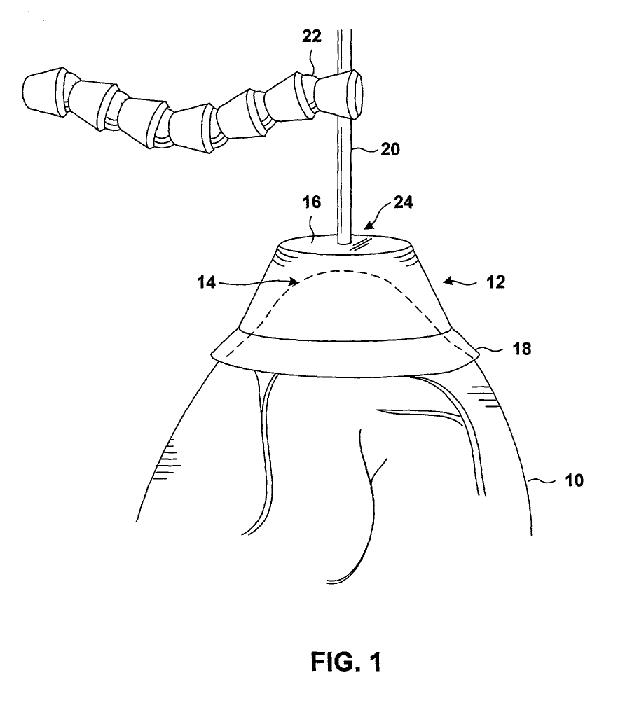

FIG. 1 is a perspective view of a heart 10, which is being held by a manipulation device 12. In the exemplary application shown in FIG. 1, a surgeon (not shown in FIG. 1) has obtained access to heart 10 and has placed manipulation device 12 over the apex 14 of heart 10. The surgeon has lifted apex 14 with manipulation device 12, giving the surgeon access to a desired region of heart 10. Although held by manipulation device 12, heart 10 has not been arrested and continues to beat. Beating causes heart 10 to move in three dimensions. In particular, heart 10 moves in translational fashion, e.g., by bobbing up and down and by moving from side to side. Heart 10 also expands and contracts as heart 10 fills with and expels blood. Heart 10 may twist as it expands and contracts.

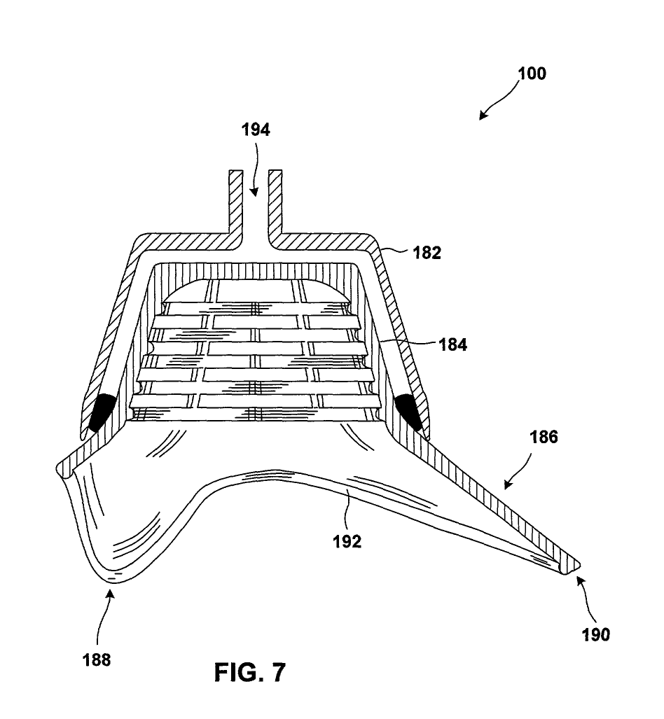

Manipulation device 12 includes an outer shell 16 and an inner shell (not shown in FIG. 1). In FIG. 1, outer shell 16 is substantially cup-shaped and symmetrical. This is an exemplary embodiment of the invention, but the invention is not limited to outer or inner shells that are cup-shaped or symmetrical. Outer shell 16 and the inner shell may take any shape. The shells may be, for example, asymmetric or irregularly shaped. The shells may, for example, include projections that extend radially outward from the centers of the shells and conform to the irregular shape of heart 10. FIG. 7 presents an example of such a shell.

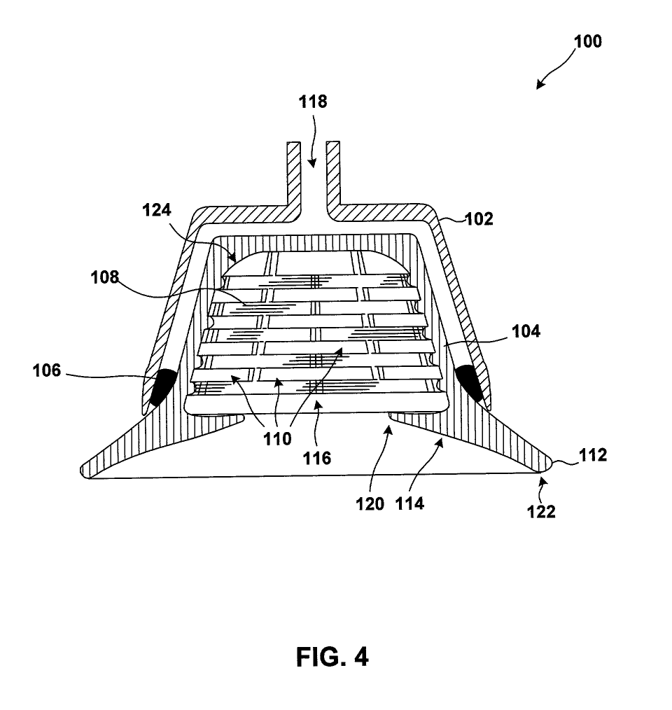

Outer shell 16 may be coupled to a skirt-like member 18 that extends distally and outward from outer shell 16. Alternatively, the inner shell may be coupled to skirt-like member 18 in a manner such as is depicted in FIG. 4. Skirt-like member 18 need not be the shape shown in FIG. 1, but may take any shape. In some embodiments, manipulation device 12 may include no skirt-like member 18 of any kind.

Skirt-like member 18 aids the adhesion between manipulation device 12 and apex 14. When vacuum pressure is supplied from a vacuum source (not shown in FIG. 1) via a vacuum tube 20, skirt-like member 18 deforms and substantially forms a seal against the surface of the tissue of heart 10. Skirt-like member 18 is typically formed of a compliant material that allows the seal to be maintained even as heart 10 beats. In particular, skirt-like member 18 may be more compliant that the remainder of manipulation device 12. As an example, skirt-like member 18 may be formed from polymeric materials, such as silicone elastomers of approximately Shore A 5 durometer.

Adherence between heart 10 and manipulation device 12 may be promoted by other factors as well, such as a tacky surface of skirt-like member 18 placed in contact with heart 10. A coating of silicone gel, for example, may be tacky and may improve the adherence between manipulation device 12 and heart 10.

Vacuum tube 20 may serve as both a support shaft for manipulation device 12 and as a supply of vacuum pressure. In an alternate embodiment, manipulation device 12 may be supported with a dedicated support shaft such as a plastic or metal shaft. In that case, vacuum tube 20 may provide little or no load-bearing capability. Instead, vacuum tube 20 may be disposed proximal to or within such a shaft. Vacuum tube 20 and/or the support shaft may be flexible.

In an operation, a surgeon may place manipulation device 12 over a portion of apex 14 of heart 10, such that heart 10 is received within the device. The surgeon may move heart 110 by moving manipulation device 12 and/or vacuum tube 20. When the surgeon has obtained access to certain areas of heart 10, the surgeon may desire to maintain heart 10 in a substantially fixed position. In an exemplary application, the surgeon may suspend heart 10 by apex 14 and hold heart 10 in place with a securing structure such as a lockable support arm 22.

Vacuum tube 20 or other support shaft may be coupled to manipulation device 12 with any coupling. Coupling 24 may be, for example, a flexible coupling that accommodates translational and rotational motion of heart 10. Various swivels and mechanical couplings may also be used.

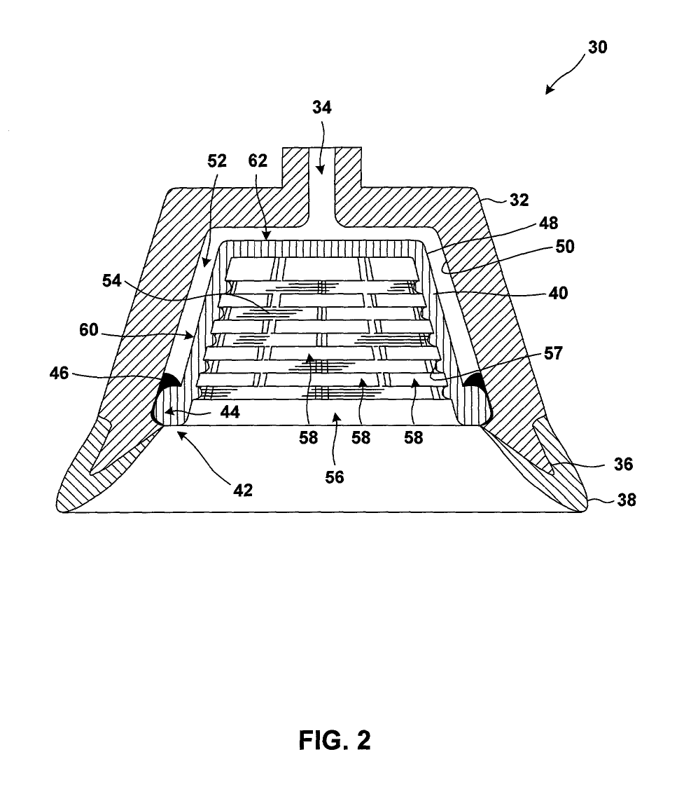

FIG. 2 is a cross-sectional side view of an exemplary manipulation device 30. Manipulation device 30 includes an outer shell 32, which provides a firm structure by which manipulation device 30 may be securely gripped by a surgeon or by an instrument. Outer shell 32 may include a structure such as a handle, knob or other attachment (not shown) for this purpose. Outer shell 32 may be formed from a variety of materials, including elastomers, metals or plastics. As an example, outer shell 32 may be formed from polymeric materials, such as silicone elastomers in the range of Shore A 30 to 75 durometer.

The proximal end of outer shell 32 defines a vacuum port 34. Vacuum pressure is conveyed to manipulation device 30 by vacuum tube 20 (not shown in FIG. 2) and is applied to manipulation device 30 via vacuum port 34. In addition, the distal end of outer shell 32 may include a mounting structure 36 that couples to a skirt-like member 38. Mounting structure 36 may be, for example, a projection to which skirt-like member 38 is adhesively bonded, or a flanged ring that mates to a groove in skirt-like member 38.

Manipulation device 30 also includes an inner shell 40. Inner shell 40 is sized to fit inside outer shell 32. Inner shell 40 is further shaped so that the distal end of inner shell 40 and the distal end of outer shell 32 may be coupled to one another. In FIG. 2, the distal end of outer shell 32 includes a shelf 42 that receives a lip 44 around the edge of inner shell 40. Inner shell 40 and outer shell 32 may be bonded at or near their distal ends, i.e., at or near the rims of the shells, by a biocompatible adhesive 46. Inner shell 40 may be formed from a different material than outer shell 32. In a typical application, inner shell 40 may be softer and more compliant than outer shell 32 so as to reduce the risk of trauma when brought in contact with the tissue. Outer shell 32 may be made of a more rigid material that imparts structural integrity to manipulation device 30 and maintains the general shape of manipulation device 30 when vacuum pressure is applied.

Most of the outer surface 48 of inner shell 40 does not rest against the inner surface 50 of outer shell 32. Rather, there is a space 52 between the outer surface 48 of inner shell 40 and the inner surface 50 of outer shell 32. Space 52 may be maintained by the respective shapes of inner shell 40 and outer shell 32. For example, as shown in FIG. 2, outer shell 32 may include a tapered inner surface 50, and lip 44 may prevent inner shell 40 from being inserted further into the tapered inner surface 50 of outer shell 32. In another embodiment, space 52 between outer shell 32 and inner shell 40 may be maintained with spacers (not shown in FIG. 2) between outer shell 32 and inner shell 40. The spacers may be molded into one or both of outer shell 32 and inner shell 40.

The inner surface 54 of inner shell 40 may be textured. When manipulation device 30 engages apex 14 of heart 10 and vacuum pressure is applied via vacuum port 34, the heart tissue tends to be drawn into the chamber 56 defined by inner shell 40. The tissue comes in contact with the inner surface 54 of inner shell 40. The texture on inner surface 54 helps grip the tissue more securely. Examples of texture may include straight lines, wavy ridges, stippling, depressions, cross-hatching or rings around inner surface 54 of inner shell 40. Inner surface 54 may have multiple textures of different sizes. In FIG. 2, for example, inner surface 54 may include ribs 57, which may themselves be textured.

In addition, inner shell 40 includes a plurality of apertures 58. In the example of FIG. 2, apertures 58 are substantially rectangular in shape. Apertures 58 provide fluid communication between space 52 and chamber 56. When manipulation device 30 engages apex 14 of heart 10 and vacuum pressure is applied via vacuum port 34, the vacuum pressure is applied to chamber 56 via apertures 58. Adhesive 46 prevents leakage of vacuum pressure at the points of connection of inner shell 40 and outer shell 32. Apertures 58 penetrate through the lateral surface 60 of inner shell 40, and may also penetrate through the proximal surface 62 of inner shell 40 as well.

Apertures 58 in the lateral surface 60 of inner shell 40 cause the organ tissue to be drawn, not merely in the direction of vacuum port 34, but in the direction of lateral surface 60. In this manner, vacuum pressure surrounds the tissue. Inner shell 40 engages the tissue with lateral surface 60, and may engage the tissue with proximal surface 62 as well. As a result, a significant fraction of the surface area of the tissue in chamber 56 comes in contact with the inner surface 54 of inner shell 40. As more surface area of the organ comes in contact with the inner surface 54 of inner shell 40, the frictional forces between the organ and the inner surface 54 of inner shell 40 increase. Frictional forces are cumulative. The frictional forces further promote adherence between the organ tissue and manipulation device 30. In addition, holding a larger surface distributes the forces over a larger area. When the forces are distributed over a larger area, less vacuum pressure may be needed to hold the organ with manipulation device 30, reducing the risk of trauma to the organ tissue and improving the adherence between the organ and manipulation device 30.

In a typical application such as the application shown in FIG. 1, the heart 10 may weigh about 6 pounds (i.e., may have a mass of about 2.7 kg, or a weight of 27 newtons). A manipulation device such as manipulation device 30 may be able to support heart 10 with a vacuum pressure of about 373 mm Hg (about 49 kilopascals). Many hospital vacuum supplies provide pressure in excess of the pressure needed to hold heart 10. These numerical values are for purposes of illustration only. The amount of pressure needed may be a function of the number of several factors such as the number of apertures, the size to the apertures, the shape of the apertures, the surface area of the manipulation device, the tackiness of the surface of the skirt-like member, the texture or textures of the inner shell and the actual weight of the organ.

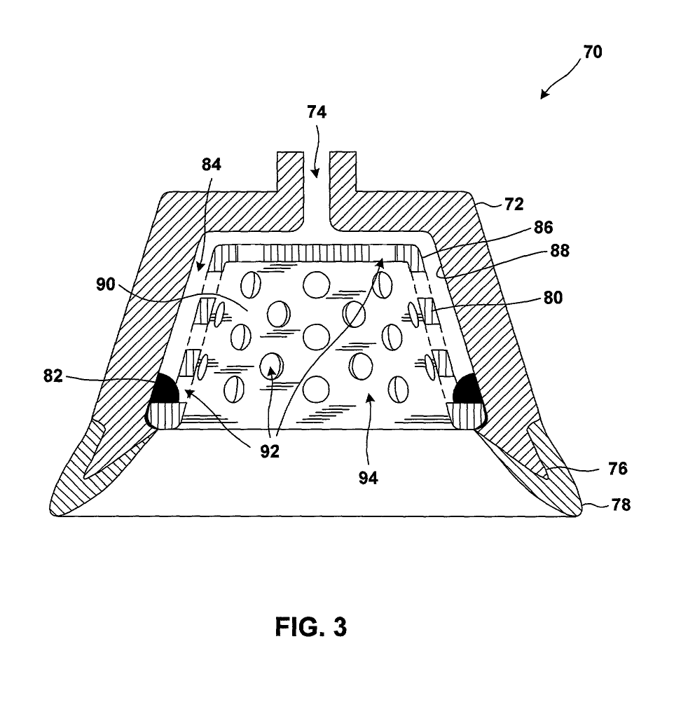

FIG. 3 is a cross-sectional side view of another exemplary manipulation device 70. Like manipulation device 30 in FIG. 2, manipulation device 70 includes an outer shell 72. Outer shell 72 defines a vacuum port 74 and includes a mounting structure 76 that couples to a skirt-like member 78. Manipulation device 70 also includes an inner shell 80 sized to fit inside outer shell 72. Inner shell 80 may be bonded to outer shell 72 by biocompatible adhesive 82. There is a space 84 between the outer surface 86 of inner shell 80 and the inner surface 88 of outer shell 72. Furthermore, the inner surface 90 of inner shell 80 may be textured.

Manipulation device 70 also includes a plurality of apertures 92. Unlike the rectangular apertures 58 of manipulation device 30 in FIG. 2, however, manipulation device 70 includes circular apertures 92. Apertures 92 provide fluid communication between space 84 and a chamber 94 defined by inner shell 80. When manipulation device 70 engages apex 14 of heart 10 and vacuum pressure is applied via vacuum port 74, the vacuum pressure is applied to chamber 94 via apertures 92. The tissue is drawn to circular apertures 92 as if each were a source of suction. As a result, a significant fraction of the surface area of the tissue in chamber 94 is brought in contact with the inner surface 90 of inner shell 80, which further promotes adherence between the tissue and manipulation device 70. In addition, the distribution of vacuum pressure over a greater surface area helps reduce the risk of trauma to heart 10.

FIG. 4 is a cross-sectional side view of another exemplary embodiment of the invention. Manipulation device 100 is similar in many respects to manipulation device 30 in FIG. 2. An outer shell 102 is bonded to an inner shell 104 with adhesive 106. The inner surface 108 of inner shell 104 may be textured, and may include as plurality of apertures 110.

Unlike manipulation device 30, however, the skirt-like member 112 of manipulation device 100 is an extension of inner shell 104, and may be formed integrally with inner shell 104. Skirt-like member 112 further comprises a canted surface 114. When an organ is inserted in chamber 116 and vacuum pressure is applied via vacuum port 118, canted surface 114 gives way and flexes such that it contacts the tissue at both inner diameter 120 and outer diameter 122, producing greater surface contact area, and promoting an effective seal. The adherence between canted surface 114 and the tissue may be enhanced by the presence of a tacky coating on canted surface 114. In addition, manipulation device 100 differs from manipulation device 30 in that inner surface 108 of inner shell 104 exhibits rounding 124 near the proximal side. Rounding 124 may better accommodate the shape of the organ.

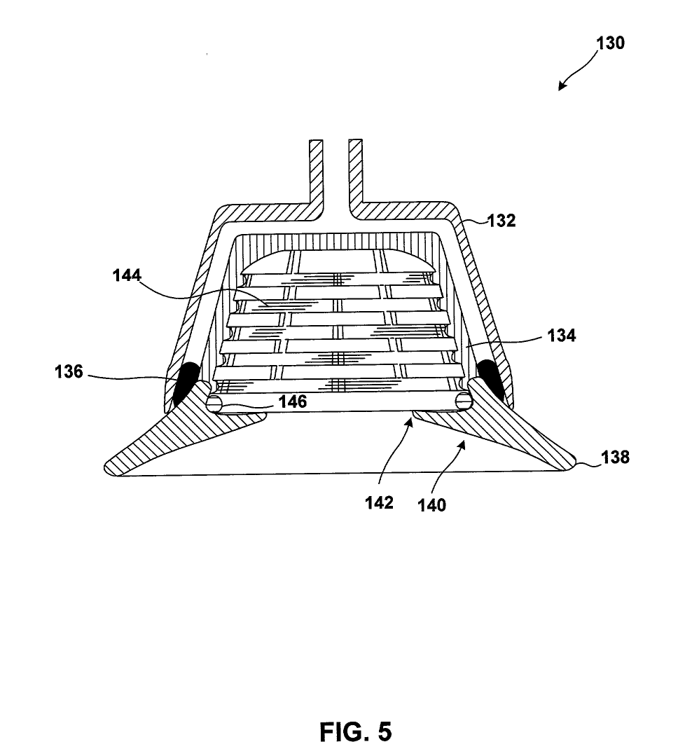

FIG. 5 is a cross-sectional side view of a further exemplary embodiment of the invention. In this embodiment, manipulation device 130 is similar to manipulation device 100 shown in FIG. 4. Manipulation device 130 includes an outer shell 132 coupled to an inner shell 134 by adhesive 136. Unlike manipulation device 100, the skirt-like member 138 is coupled to inner shell 134, but is not an extension of inner shell 134. In other words, skirt-like member 138 may have characteristics different from inner shell 134. Skirt-like member 138 may be constructed of a more pliable material than inner shell 134, for example, or may have a more tacky quality than inner shell 134.

Like skirt-like member 112 of manipulation device 100, skirt-like member 138 comprises a canted surface 140. When skirt-like member 138 comprises a very pliable material, inner diameter 142 may tend to buckle and rest against inner surface 144. When inner diameter 142 buckles, it may be more difficult to establish a seal with the organ tissue. A supporting structure such as an O-ring 146 may be bonded to inner shell 134, skirt-like member 138 or both to prevent buckling. O-ring 146 may also help improve the contact between skirt-like member 112 and the organ tissue when vacuum pressure is applied. O-ring 146 may be formed, for example, from polymeric materials, such as silicone elastomers of approximately Shore A 75 durometer. O-ring 146 may be bonded to inner shell 134, skirt-like member 138 or both with the same type of biocompatible adhesive 136 that couples outer shell 132 to inner shell 134.

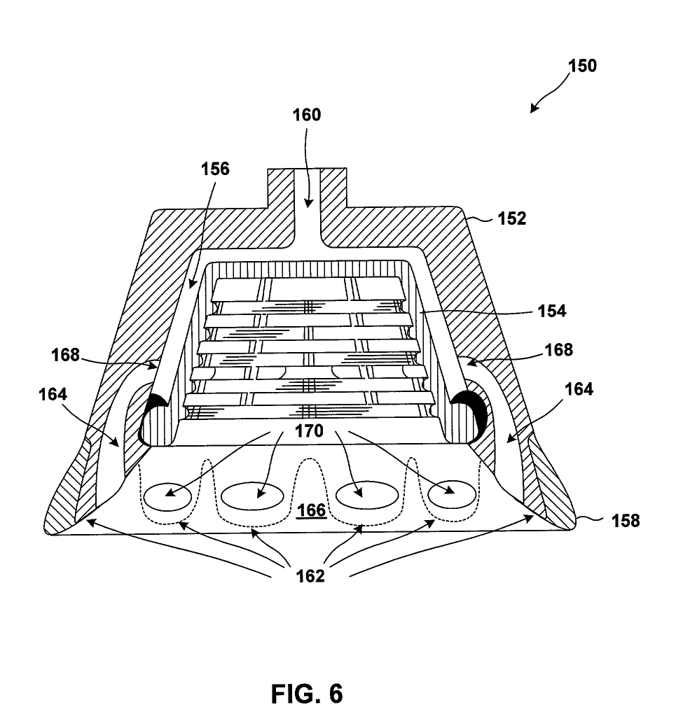

FIG. 6 is a cross-sectional side view of an exemplary manipulation device 150 that is similar to manipulation device 30 shown in FIG. 2. In particular, manipulation device 150 includes an outer shell 152 and an inner shell 154. Space 156 separates outer shell 152 from inner shell 154. The distal end of outer shell 152 may be coupled to a skirt-like member 158. The proximal end of outer shell 152 may include a vacuum port 160.

Outer shell 152 of manipulation device 150 includes a set of finger-like extensions 162 that extend distally from outer shell 152. Extensions 162 may be integrally formed with outer shell 152 by molding. Skirt-like member 158 may surround and/or extend below extensions 162. Extensions 162 may thin in both thickness and width as they approach the lower extent of skirt-like member 158.

Extensions 162 may serve several purposes. First, extensions 162 may provide added support to manipulation device 150, in particular, support to resist collapse of skirt-like member 158 under vacuum pressure. Skirt-like member 158 may be formed from a substantially compliant material, such as a silicone elastomer of approximately Shore A 5 to 10 elastomer. Alternatively, skirt-like member 158 may be formed from a silicone gel such as Nu-Sil MED 6340 that is both compliant and tacky, enhancing sealing pressure. When skirt-like member 158 is supported by extensions 162, skirt-like member 158 may be more flexible than in other embodiments of the invention.

In addition, extensions 162 may enclose and provide structural support for channels 164. Channels 164 provide fluid communication between space 164 and chamber 166. Channels 164 are bounded by proximal ports 168 and distal ports 170. In the embodiment shown in FIG. 6, proximal ports 168 and distal ports 170 are oval-shaped, but the invention encompasses ports of any shape. When manipulation device 150 engages apex 14 of heart 10 and vacuum pressure is applied via vacuum port 160, the tissue is drawn to distal ports 170 as if each were a source of suction. In this way, channels 164 enhance adherence between the tissue and skirt-like member 158 and/or between the tissue and extensions 162, and may enhance the seal between the tissue and manipulation device 150 as well.

Channels 164 may provide additional security against unintended release of the organ from manipulation device 150. An organ may disengage from a manipulation device when the load of the organ exceeds the load that can be lifted by the device, or an organ may disengage from a manipulation device when the tissue separates or delaminates from the sides of the inner shell. When the tissue delaminates, the adhesive seal is damaged and a rapid disengagement may follow. In the case of a heart, delamination may occur when the heart distends along the lifting axis while filling and expelling blood. Channels 164 cooperate to hold the sides of manipulation device 150 against the tissue, reducing the risk of delamination and rapid release of the organ.

FIG. 7 is a cross-sectional side view of an exemplary manipulation device 180 that is similar to manipulation device 100 shown in FIG. 4. In particular, manipulation device 180 includes an outer shell 182 and an inner shell 184, with a skirt-like member 186 being an extension of inner shell 184. Skirt-like member 186 may be formed integrally with inner shell 184.

Skirt-like member 186 in FIG. 7 includes protrusions 188, 190 that extend radially outward from the center of inner shell 184. Although FIG. 7 shows two protrusions 188, 190, any number of protrusions may be applied. The protrusions may be substantially equidistant from each other, or may be irregularly spaced. The protrusions may be of equal length and width, or may have dimensions different from one another. Protrusions 188, 190 may help manipulation device 180 conform to the shape of the organ. When manipulation device 180 is positioned over apex 14 of heart 10, for example, protrusions 188, 190 conform to the irregular shape of heart 10. In addition, protrusions 188, 190 increase the surface area of the organ that may be held by manipulation device 180, thereby improving the robustness of the adherence between manipulation device 180 and the organ.

Skirt-like member 186 may also include a lip-like edge 192 around the distal rim of skirt-like member 186. When vacuum pressure is applied via vacuum port 194, edge 192 may contribute to the formation of a more secure seal. Edge 192 may include a relatively flat surface that facilitates contact between edge 192 and the organ.

As FIGS. 2 through 7 demonstrate, the inner and outer shells may be of any shape. The inner shell may include apertures of any shape and in any pattern. The skirt-like member may be constructed in any number of ways, or may be omitted in its entirety. The invention encompasses all of these variations.

In various embodiments of the invention, The number, size and shape of apertures may vary. In general, the number of apertures is a function of the hardness and flexibility of the inner shell. Larger apertures may be desirable to draw the tissue to the apertures, but larger apertures may lead to deformation of the inner shell during use. Making the inner shell stronger to resist deformation may affect the flexibility of the inner shell, and may increase the risk of trauma to the tissue.

The number and size of apertures may further be a function of the overall dimensions of the manipulation device. A manipulation device may include a chamber about 1.5 inches (3.8 cm) in diameter at the widest point, with a surface area of about 3.8 square inches (24.5 square centimeters). An inner shell with these dimensions may accommodate, for example, about thirty round apertures with a diameter of about 0.25 inches (0.64 centimeters) or about 150 rectangular apertures of 0.005 inches by 0.2 inches (0.13 centimeters by 0.5 centimeters). The dimensions of the manipulation device may be vary, and a manipulation device having a larger surface area may accommodate more apertures than a manipulation device having a smaller surface area. Further, as noted above, the number, size and shape of apertures may depend upon the structural characteristics of the inner shell.

The invention can provide one or more advantages. The inner shell provides a large surface that contacts and grips the organ, holding it securely. The softness and compliance of the inner shell, however, protects the organ from damage. The plurality of apertures act like individual suction sources, drawing the tissue into and against the inner shell. Increased contact between the inner shell and the organ reduces the risk that the organ will accidentally slip out of the manipulation device. The texture of the inner surface of the inner shell may also contribute to prevention of slippage.

In the case of heart surgery, the heart can be manipulated and securely held in place so that the surgeon may have access to a desired region of the heart. Because of the adherence between the manipulation device and the heart, the heart is less likely to be dropped by the manipulation device. The heart may be simultaneously granted translational and rotational freedom so that the hemodynamic functions of the heart are maintained, and so that the patient is less likely to suffer from circulatory problems during surgery. In addition, the manipulation device may be coupled to a vacuum tube or support shaft using any of several flexible or other movable couplings that accommodate the motion of the heart.

A further advantage is that the manipulation device may be customized to the needs of the patient. For example, the inner shell may be sized and/or shaped to better accommodate the size and shape of the heart of a particular patient.

Various embodiments of the invention have been described. These embodiments are illustrative of the practice of the invention. Various modifications may be made without departing from the scope of the claims. For example, the vacuum port need not be centered on the proximal side of the manipulation device as shown in the figures, and the shells need not have a cup-like shape. The apertures in the inner shell need not be uniformly sized or uniformly shaped.

Moreover, features from some embodiments discussed herein may be incorporated into other embodiments. Manipulation device 150 shown in FIG. 6, for example, may include extensions that extend from inner shell 154 rather than from outer shell 152, or extensions may extend distally from both shells. Skirt-like member 158 may include a canted surface, which may be supported with a supporting structure such as an O-ring. Manipulation device 180 shown in FIG. 7 may include a skirt-like member that is coupled to outer shell 182. The invention encompasses all of these variations.

There are advantages to having the outer and inner shells made from different materials. The outer shell is generally more rigid than the inner shell, imparting structural integrity to the device, while the inner shell is generally softer and less irritating to the organ tissue. The invention encompasses devices, however, in which the inner and outer shells are composed of the same material. The invention also encompasses devices in which the inner and outer shells are molded or formed integrally from a single piece of material.

The invention encompasses manipulation devices having textured inner shells and manipulation devices with inner shells having a smooth inner surface. The inner surface of the inner shell may be coated with a tacky material, such as soft silicone gel, which may further promote adherence between the organ and the inner shell. These and other embodiments are within the scope of the following claims.

1. An organ manipulation device comprising:
an outer shell; and
an inner shell disposed within the outer shell, the inner and outer shells defining a space, the inner shell defining a chamber and including an aperture in fluid communication with the space and the chamber.

2. The device of claim 1, the outer shell further comprising a vacuum port in fluid communication with the space.

3. The device of claim 1, wherein the inner shell has a generally cup-like shape.

4. The device of claim 1, further comprising a skirt-like member coupled to the outer shell that extends outward from the outer shell.

5. The device of claim 1, further comprising a skirt-like member coupled to the inner shell that extends outward from the outer shell.

6. The device of claim 5, wherein the skirt-like member is formed integrally with the inner shell.

7. The device of claim 1, further comprising a skirt-like member coupled to at least one of the outer shell and the inner shell, the skirt-like member including a canted surface.

8. The device of claim 7, further comprising a supporting structure proximal to an inner diameter of the skirt-like member.

9. The device of claim 8, wherein the supporting structure is an O-ring.

10. The device of claim 1, wherein the aperture has a substantially rectangular shape.

11. The device of claim 1, wherein the aperture has a substantially circular shape.

12. The device of claim 1, wherein the inner shell includes a plurality of apertures in fluid communication with the space and the chamber.

13. The device of claim 12, wherein the plurality of apertures are distributed about the inner shell.

14. The device of claim 1, wherein the inner shell includes an inner surface, and wherein the inner surface includes a texture.

15. The device of claim 14, wherein the texture comprises ribs.

16. The device of claim 1, further comprising an adhesive that bonds the inner shell to the outer shell.

17. The device of claim 1, wherein the inner shell includes a lip, and wherein the outer shell includes a shelf that receives the lip.

18. The device of claim 1, further comprising an extension that extends distally from at least one of the outer shell and the inner shell.

19. The device of claim 18, wherein the extension encloses a channel in fluid communication with the space and the chamber.

20. The device of claim 1, further comprising a skirt-like member coupled to at least one of the outer shell and the inner shell, the skirt-like member including a protrusion that extends outward from the inner shell.

21. A method comprising:
constructing an inner shell that defines a chamber, the inner shell sized and shaped to receive an organ and to fit inside an outer shell;
providing an aperture in the inner shell; and
coupling the inner shell to the outer shell to define a space, the aperture in fluid communication with the space and the chamber.

22. The method of claim 21, further comprising constructing the outer shell.

23. The method of claim 21, wherein coupling the inner shell to the outer shell comprised coupling the inner shell to the outer shell with an adhesive.

24. The method of claim 21, wherein the aperture has a rectangular shape.

25. The method of claim 21, wherein the aperture has a circular shape.

26. The method of claim 21, wherein constructing the inner shell comprises constructing a textured surface on the inner shell.

27. The method of claim 21, further comprising coupling a skirt-like member to the outer shell.

28. The method of claim 21, further comprising coupling a skirt-like member to the inner shell.

29. The method of claim 21, wherein constructing the inner shell comprises extending a distal edge of the inner shell to form a skirt-like member.

30. The method of claim 21, further comprising:
providing an extension distally from at least one of the outer shell and the inner shell; and
coupling a skirt-like member to the extension.

31. The method of claim 21, further comprising:
constructing the inner shell from a first material; and
constructing the outer shell from a second material.

32. A method comprising:
receiving an organ in a chamber defined by an inner shell;
applying vacuum pressure to an outer shell coupled to the inner shell, the outer shell and the inner shell defining a space; and
applying vacuum pressure to the chamber via an aperture in fluid communication with the space and the chamber.

33. The method of claim 32, further comprising manipulating the organ by manipulating the outer shell.

34. The method of claim 32, wherein receiving the organ in the chamber defined by the inner shell comprises engaging an apex of a heart with the inner shell.
